(19) United States
(12) Patent Application Publication
(10) Pub. No.: US 2008/0038237 A1
(43) Pub. Date: Feb. 14, 2008

(54) Trojan horse immunotherapy

(76) Inventors: Robert M. Sapolsky, San Francisco, CA (US); Nathan C. Manley, Stanford, CA (US); Sheila Brooke, Sunnyvale, CA (US); Klaus M. Dinkel, Berlin (DE); Carolina D. Munhoz, Sao Paolo (BR); Angela L. Riepel, Sunnyvale, CA (US); Andrea C. Nicholas, San Ramon, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/713,218

(22) Filed: Feb. 28, 2007

Related U.S. Application Data
(60) Provisional application No. 60/777,843, filed on Feb. 28, 2006.

(51) Int. Cl. A61K 48/00; A61P 25/00; A61P 37/00

(52) U.S. Cl. 424/93.21

(57) ABSTRACT

The present invention provides methods for the delivery of therapeutic proteins to an area of inflammation and neuronal damage within the central nervous system (CNS). In this invention, immunocytes that can cross the blood brain barrier and access sites of CNS injury/inflammation, are genetically engineered to express and secrete a therapeutic protein of interest and are transplanted into a subject having a CNS injury. These genetically modified “carrier immunocytes” home to the site of a CNS lesion and secret the therapeutic agent, thereby ameliorating disease symptoms and pathology.

Neurological diseases can be devastating pathological conditions, with resultant damage to brain tissue that significantly compromises the CNS and leads to both physical and mental impairments. Neurological diseases can arise from acute conditions, such as stroke or seizure, or from long-term neurodegeneration, as is the case in Alzheimer's and Parkinson's disease. Both forms of neurological disease cause a substantial loss of brain cells, including neurons. These debilitating conditions lack effective treatment options.

Understanding of neuronal damage and loss due to neurological disease and injury has increased significantly during the past several decades, including an increase in our understanding of the molecular pathways that control neuron death. In tissue culture and animal models of neurological insults, delivery and enhanced expression of certain neuroprotective genes or of the encoded protein products themselves, can significantly reduce the extent of neuron death in these conditions.

The clinical applicability of therapeutic strategies based on these research findings is hindered, however, by the difficulty of delivering target genes or proteins to the CNS. Specifically, gene or protein delivery to a site of injury in the CNS is hindered because access to brain tissue from the circulation is restricted by the ‘blood brain barrier’ (BBB). To circumvent the BBB, injury-specific gene or protein delivery to the CNS can be achieved via direct injection of the gene or protein therapeutic agent into brain tissue. This is highly invasive, requiring drilling a hole in the skull and controlled penetration of the brain with a syringe. In addition, it is difficult, if not impossible, to deliver the gene or protein therapeutic agent to all of the affected sites. As an alternative, the gene or protein therapeutic agent can be introduced into the blood stream wherein it migrates to the site of injury in the brain. This approach is sub-optimal because only a small percentage of the gene or protein therapeutic agent arrives at the injury site; the vast majority ends up elsewhere in the body and may lead to unwanted side effects. Moreover, techniques that open up the BBB are often damaging in and of themselves, and cause non-specific opening, resulting in delivery of the therapeutic agent throughout the brain, often with untoward side effects.

There is substantial research and clinical interest in the ability to deliver genetic sequences and proteins to treat CNS diseases and injury. For such methods, transport is preferably non-invasive, appropriately localized, and within an appropriate time window for neuroprotection. The present invention is drawn to meeting these, and other, needs.

### SUMMARY OF THE INVENTION

Compositions and methods are provided for a genetic or protein delivery system that preferentially targets injured neural tissue, called Trojan Horse Immunotherapy (THI). A primary application of THI is the delivery and expression of therapeutic biomolecules to an injured area within the central nervous system (CNS). THI employs genetically engineered immunocytes, which include without limitation dendritic cells (DCs), to deliver a therapeutic molecule of interest to the site of injury in the CNS, exploiting the ability of such cells to cross the blood brain barrier (BBB) and access sites of injury and inflammation in the CNS when injected into the bloodstream of a subject.

### BRIEF DESCRIPTION OF THE DRAWINGS

FIG. 1: Fluorescently-labeled DCs migrate from the tail vein to the hippocampus in response to a KA-induced lesion. (A) Bright field image of a hippocampal section from a DC-injected animal. The white arrow shows the KA infusion site, and the black box indicates the region in (B) and (C). Abbreviations: DG=dentate gyrus, CA=Comu Ammonis. Magnification=40×. (B) Close-up of the area between DG and CA3 for a DC-injected animal. Prior to delivery via the tail vein, cultured DCs were labeled with PKH26 cell dye (Sigma), which causes fluorescent emission at 570 nm. DCs were injected 18 hours after KA infusion, and the image was taken 6 hours after DC delivery. The inset shows a fluorescently-labeled DC in culture, taken at the same time as the larger image. Magnification=200×. (C) Fluorescent image of the lesioned hippocampus from a vehicle-injected animal. Autofluorescence is detectable along the DG, but is distinguishable from DC signal present in (B). Magnification=200×.

FIG. 2: Schematic representation of the modified lentiviral transfer vectors. Constructs contain the reporter gene, eGFP, (A), or the neurotrophin, BDNF, and eGFP separated by an IRES (B). Abbreviations: CMV=cytomegalovirus promoter; eGFP=enhanced green fluorescent protein; hBDNF=human brain-derived neurotrophic factor; IRES=internal ribosomal entry site; LTR=long terminal repeat; Ψ=packaging signal; Δgag=frame-shifted gag gene; RRE=rev-responsive element; trip=central polypurine tract+termination sequence; gpt=xanthine-guanine phosphoribosyltransferase gene; SV40 prom-enh=simian virus enhancer/promoter; SSS=secretory signal sequence.

### DETAILED DESCRIPTION OF THE EMBODIMENTS

Genetically engineered immunocytes are used to deliver a therapeutic molecule of interest to a site of a lesion in the CNS. To achieve lesion-specificity, THI takes advantage of local pro-inflammatory signals produced by damaged neurons. The inflammatory signals, e.g., cytokines, chemokines, etc. act as a homing beacon br immune cells, which are stimulated to infiltrate and survey the injury site. In addition, signal production in the CNS causes localized opening of the blood brain barrier (BBB), which signal facilitates entry of immune cells into damaged brain tissue. Because the BBB remains intact elsewhere in the CNS, immune cells are only permitted entry at the site of injury. Lesions, as used herein, include acute injuries and other localized sources of inflammatory cytokines, e.g. certain chronic diseases, etc.

In the methods of the invention, inflammation-responsive immune cells are genetically modified ex vivo or in vivo to express a genetic sequence of interest, usually a genetic sequence encoding a therapeutic protein. The immunocytes may be allogeneic or autologous. The immunocytes are administered to a patient that is susceptible to, orsuffering from a CNS lesion, e.g. acute injury, etc. The immunocytes are targeted to the specific site of injury, where they are able to cross the BBB and infiltrate the damaged brain tissue.

The method of the invention may encompass the following steps. Immunocytes or precursors thereof are isolated from an autologous or allogeneic source, e.g. from peripheral blood, lymph nodes, bone marrow, etc. and maintained or cultured ex vivo. A “gene cargo” encoding a therapeutic agent is introduced into the cultured cells. Vectors of interest for the gene cargo include viral vectors, e.g. lentiviral vectors, adenovirus vectors, etc. The cells harboring the gene cargo (also called “carrier” cells) are administered to the subject, e.g. by i.v. administration. After introduction into the bloodstream of the subject, the carrier cells migrate to the CNS injury site, cross the BBB into damaged brain tissue and express their therapeutic gene cargo (e.g., a secreted protein).

As used herein, the term “immunocyte” refers to an immune competent cell, e.g. T cells, particularly Th1, Th2, and T reg cells; antigen presenting cells, including B cells, dendritic cells; neutrophils; macrophage; natural killer cells, etc. Preferred cells lack killing or inflammatory activity, e.g. dendritic cells, B cells, etc. Such immunocytes are competent for “trafficking”, as known in the art, wherein the cells respond to signaling by chemokines or cytokines by migrating to the site of signaling, then invading the vascular endothelium and crossing into the extravascular space.

In one embodiment of the invention, the immunocyte is a dendritic cell. As used herein, dendritic cell (DC) refers to any member of a diverse population of morphologically similar cell types found in lymphoid or non-lymphoid tissues. DCs are referred to as “professional” antigen presenting cells, and have a high capacity for sensitizing MHC-restricted T cells. DCs may be recognized by function, by phenotype and/or by gene expression pattern, particularly by cell surface phenotype. These cells are characterized by their distinctive morphology, high levels of surface MHC-class II expression and ability to present antigen to CD4⁺ and/or CD8⁺ T cells, particularly to naïve T cells (Steinman et al. (1991) Ann. Rev. Immunol. 9:271; incorporated herein by reference for its description of such cells).

The cell surface of DCs is unusual, with characteristic veil-like projections, and is characterized by expression of the cell surface markers CD11c and MHC class It. Most DCs are negative for markers of other leukocyte lineages, including T cells, B cells, monocytes/macrophages, and granulocytes. Subpopulations of dendritic cells may also express additional markers including 33D1, CCR1, CCR2, CCR4, CCR5, CCR6, CCR7, CD1a-d, CD4, CD5, CD8alpha, CD9, CD11b, CD24, CD40, CD48, CD54, CD58, CD80, CD83, CD86, CD91, CD117, CD123 (IL3Rα), CD134, CD137, CD150, CD153, CD162, CXCR1, CXCR2, CXCR4, DCIR, DC-LAMP, DC-SIGN, DEC205, E-cadherin, Langerin, mannose receptor, MARCO, TLR2, TLR3 TLR4, TLR5, TLR6, TLR9, and several lectins. The patterns of expression of these cell surface markers may vary along with the maturity of the dendritic cells, their tissue of origin, and/or their species of origin.

Immature DCs express low levels of MHC class II, but are capable of endocytosing antigenic proteins and processing them for presentation in a complex with MHC class II molecules. Activated DCs express high levels of MHC class II, ICAM-1 and CD86, and are capable of stimulating the proliferation of naïve allogeneic T cells, e.g. in a mixed leukocyte reaction (MLR).

Functionally, DCs may be identified by any convenient assay for determination of antigen presentation. Such assays may include testing the ability to stimulate antigen-primed and/or naïve T cells by presentation of a test antigen, followed by determination of T cell proliferation, release of IL-2, and the like.

DCs are useful for the delivery of therapeutic agents because of their ability to migrate to sites of inflammation, as well as their capacity for transgene uptake and expression. As indicated above, DCs are professional antigen presenting cells that migrate throughout the body in an immature state via the vascular and lymphatic systems. In response to inflammatory ligands present at a site of injury or infection, immature DCs undergo an activation process called diapedesis and extravasate into the damaged tissue. Importantly, DCs are involved in the earliest stages of immune response to injury or infection, due in part to their constitutive expression of MHC class II surface molecules.

The source of DCs and their subsequent manipulation will depend on the specific nature of the THI being employed. As such, in certain embodiments of the invention, DCs of interest are autologous, meaning that they derived form the subject to be treated with THI. In other embodiments, the DCs are from a donor (i.e., allogeneic). In certain of these embodiments, the allogeneic DCs are from a compatible donor, i.e., HLA typed so that they are histocompatible with the subject into which they will be transplanted.

In certain embodiments, DCs for use in the invention are derived from lymphoid tissue, including bone marrow, blood, mobilized peripheral blood, spleen, lymph node, and cord blood. The DCs obtained from the lymphoid tissue can be in a variety of developmental states, from immature DC precursors to mature DCs. In embodiments in which immature DC precursors are isolated, they may be differentiated into mature DC in vitro using any number of culture conditions, including with specific growth factors (e.g., IL4, GCSF, Flk-2 ligand, etc. (for review, see European Cytokine Network. 2002 April-June; 13(2):186-99; incorporated herein by reference).

In certain embodiments, DCs are enriched or isolated as is known in the art. In certain of these embodiments, DCs are enriched based on the cell surface expression of specific molecules (e.g., those noted above) using marker-specific monoclonal antibodies (e.g., sorting by flow cytometry, immunobead selection, immunopanning, etc.)

In certain embodiments, the DCs or DC precursors are frozen in liquid nitrogen (or equivalent) prior to use in THI. For example, the tissue source of the DCs (e.g., bone marrow) may be harvested, frozen and stored until needed. Likewise, DCs derived in vitro from precursors can be frozen and stored until used for THI. If frozen, the cells will usually be stored in a 10% DMSO, 50% FCS, 40% RPMI 1640 medium in liquid nitrogen.

### Gene Delivery Vectors

Therapeutic proteins, which are discussed in detail below, are delivered to the site of inflammation in THI by introducing a gene delivery vector encoding the therapeutic protein into an immunocyte and administering such “carrier” immunocytes to the host.

The gene delivery vectors of the present invention include a gene expression cassette, which provides all of the genetic sequence required for expression of the protein of interest, including coding sequences, transcriptional regulatory sequences, translational regulatory sequences, and the like, when present in the immunocyte. The gene expression cassette will generally be comprised within a vector, where the vector contains a suitable origin of replication, and such genes encoding selectable markers as may be required for growth, amplification and manipulation of the vector, prior to its introduction into the recipient. Suitable vectors include plasmids, YACs, BACs, bacteriophage, viral, and the like, including retrovirus, adenovirus, adeno associated virus, lentivirus, etc. In certain embodiments, lentiviral vectors are employed.

The expression cassette includes a promoter, usually an exogenous transcriptional initiation region, i.e. a promoter other than the native promoter, which is functional in the targeted cells. The promoter may be introduced by recombinant methods in vitro, or as the result of homologous integration of the sequence by a suitable host cell. The promoter is operably linked to the coding sequence to produce a translatable mRNA transcript. Expression vectors conveniently will have restriction sites located near the promoter sequence to facilitate the insertion of therapeutic gene sequences. Many strong promoters for mammalian cells, including immunocytes, are known in the art, including the β-actin promoter, SV40 early and late promoters, human cytomegalovirus promoter, retroviral LTRs, etc. The promoters may or may not be associated with enhancers, where the enhancers may be naturally associated with the particular promoter or associated with a different promoter.

In certain embodiments, the promoter is regulatable, such as the Tet-ON system, where expression of the gene of interest is dependent on the addition of doxycycline. There are a number of other regulatable expression systems, including those dependent on a variety of exogenous or endogenous activators, including chemical activators, hormones and steroids, e.g., corticosteroids.

In certain embodiments, transgene expression is linked to arrival of transduced immunocytes to the site of CNS injury as a means to minimize unwanted transgene expression elsewhere in the recipient organism. In one such embodiment, the promoter is an NFκB-responsive promoter. NFκB is a general term used to describe a number of dimeric combinations of members of the Rel family of gene regulatory proteins that possess transcriptional activating properties. The most common form of NFκB consists of a heterodimer of p50 (NFκB1) and p65 (RelA) proteins. This complex has the ability to bind with promoter sequences in DNA and to inaugurate transcription for many proinflammatory mediators. However, other combinations of Rel family members have been identified, and different configurations of Rel proteins (such as p65/p50 and p65/p52) may have preferential sensitivities to different target promoter sequences. In unstimulated cells, NFκB is retained in the cytoplasm through interactions with inhibitory proteins of the inhibitory factor kappa B (IκB) family. Degradation of IκB leads to activation of NFκB, which is defined as translocation of the NFκB complex from the cytoplasm to the nucleus. Once in the nucleus, NFκB binds specific promoter elements of DNA and induces transcription of relevant genes. The specificity of NFκB for DNA promoter segments is dependent on nucleotide base sequences recognized by NFκB. Promoters under the control of NFκB include the proinflammatory cytokines TNFα and IL-1 (Coilart et al. (1990) Mol. Cell Biol. 10:1498-1506; Hiscott et al. (1993) Mol. Cell. Biol. 13:6231-6240), numerous chemokines, such as MIP-1α, MIP-1β, etc. (Widmer et al. (1993) J. Immunol. 150:4996-5012), and vascular endothelial cell adhesion molecules (Collins et al. (1995) FASEB J. 9:899-909), the teachings of which references are herein specifically incorporated by reference for the disclosure of NF-κB regulatable promoters.

A termination region is provided 3′ to the coding region, where the termination region may be naturally associated with the gene of interest or may be derived from a different source. A wide variety of termination regions may be employed without adversely affecting expression.

In addition to an expression cassette for the therapeutic gene of interest, in certain embodiments the vector includes an expression cassette for a marker gene. The marker gene provides a way to identify immunocytes that contain the vector and in certain embodiments be used to isolate or purify immunocytes that contain the vector. Marker genes of interest include genes that encode any of a variety of known marker proteins, including, but not limited to, cell surface expressed proteins, fluorescent proteins (e.g., green fluorescent protein (GFP) and the like), and metabolic proteins (e.g., β-galactosidase). The marker gene can be expressed from a distinct promoter in the vector or be expressed in tandem with the therapeutic gene of interest via linkage through internal ribosome entry site (IRES).

In certain embodiments, the vector includes a gene expression cassette for genes that aid in homing of immunocytes to site of injury in the CNS. For example, targeting may be enhanced for hippocampal regions by overexpression of an inflammatory-responsive chemokine receptor, e.g. CCR5 and the like. Overexpression of CCR5 also may extend the duration of immunocyte localization to the hippocampal lesion. Other chemokine receptors of interest include receptors for monocyte chemoattractant protein-1 (MCP-1) and macrophage inflammatory protein-1α (MIP-1α), and receptors for β-chemokines , e.g. MCP-2 [CCL8], MCP-3 [CCL7], and MCP4 [CCL13] The homing gene expression cassette can be engineered into the gene therapeutic vector in a similar fashion as the marker gene (e.g., with its own promoter or via an IRES).

In certain embodiments, individual gene expression cassettes are present on the same vector whereas in other embodiments, the expression cassettes are on distinct vectors.

In certain embodiments, more than one therapeutic gene of interest is present in the vector or vectors.

The various manipulations of the vector can be carried out in vitro or may be performed in an appropriate host, e.g. E. coli. After each manipulation, the resulting construct may be cloned, the vector isolated, and the DNA screened or sequenced to ensure the correctness of the construct. The sequence may be screened by restriction analysis, sequencing, or the like.

The gene therapeutic expression construct may be introduced into immunocytes by any number of routes, including transfection, viral infection, microinjection, or fusion of vesicles.

Therapeutic Gene Cargo

The DNA sequence of the therapeutic gene cargo, i.e., the therapeutic gene(s) of interest, in the gene therapeutic vectors of the invention may include a native gene, a chimeric gene, encode a tagged or fusion protein, or may differ from a native sequence by the deletion, insertion or substitution of one or more nucleotides, provided that they encode a protein with the desired biological activity. Similarly, genetic sequences may be truncated or extended by one or more nucleotides. Alternatively, DNA sequences suitable for the practice of the invention may be degenerate sequences that encode the protein of interest. DNA sequences of the invention may have at least 70%, at least 80%, at least 90%, at least 95% or at least 99% sequence identity to a native coding sequence. They may originate from any species, though DNAs encoding human proteins are preferred. Variant sequences may be prepared by any suitable means known in the art. Proteins of the invention that differ in sequence from naturally occurring proteins may be engineered to differ in activity from the naturally occurring protein. Such manipulations will typically be carried out at the nucleic acid level using recombinant techniques, as known in the art.

In THI, the immunocytes are used as vehicles for delivering therapeutic sequences to sites of CNS injury. The proteins may be secreted from the immunocytes so that they act on the target cells (e.g., neurons) at the injury site. As such, in certain embodiments of the invention, the gene therapeutic vectors incorporate a secretory signal sequence to ensure that the expressed protein is secreted by carrier immunocytes into the extracellular space of the injury site. Appropriate protein secretion relies on the presence of an N-terminal secretory signal sequence, incorporated in the construct design of the transfer vector (FIG. 2B). In certain embodiments, the signal sequence is derived from the native gene of interest, whereas in other embodiments, a signal sequence from a heterologous gene is employed.

Certain proteins, such as anti-inflammatory cytokines, growth factors or antioxidants, can protect neurons when present in the extracellular space. In such instances the presence of a secretory signal sequence is sufficient for their delivery. In other embodiments, the protein is protective when internal to a neuron, e.g. when the protein is an antioxidant that localizes to an intracellular organelle. In these embodiments, the immunocytes are engineered to secrete a protein that is taken up by damaged neurons. In certain of these embodiments, this will be accomplished by modifying the therapeutic gene of interest to include a protein transduction domain (PTD), which facilitates uptake of the corresponding protein by the recipient's neurons. PTDs of interest include those based on the PTD of the Tat protein of HIV as well as other synthetic or naturally occurring PTDs known in the art.

In certain embodiments, the therapeutic gene is engineered to contain an antigenic tag, a number of which are known in the art (e.g., FLAG, HIS, Myc, HA, etc.). This allows for detection of the expression of the protein of interest from the vector both in vitro and in vivo.

Below is a discussion of representative therapeutic proteins that find use in THI. This list not exhaustive, as other therapeutic genes also find use in THI. A more complete discussion of the genes listed below can be found in Nature Reviews: Neuroscience (2003) vol. 4(1) pp 61-69, incorporated herein by reference. Proteins of interest also include antibodies, e.g. single chain antibodies, Fab fragments, etc., which specifically bind to a targeted molecule, e.g. to inhibit a specific protein of interest, to activate a receptor, and the like. Antibody coding sequences maybe present as one, two or more distinct sequences, usually a heavy chain and a light chain sequence, although in some embodiments a single chain antibody is produced.

Proteins that target inflammatory elements of acute injury. Neuronal necrosis, which causes the cells to rupture and release their constituents into the extracellular space, activates complement cascades and neutrophil infiltration, and releases cytokines and ROS. This inflammation can cause secondary damage to otherwise healthy neighbouring neurons. As such, delivery of proteins that block the inflammatory actions of the cytokine interieukin-1 (IL-1) (e.g., an IL-1 receptor antagonist) are of interest in the subject invention. It has been reported that this IL-1 antagonist expression decreases infarct and excitotoxic damage to the striatum and hippocampus, and also decreases neutrophil infiltration.

Neurotrophins. Neurotrophins are well-known for their role in compensatory sprouting of dendritic processes following neuronal injury, but are less well-known for their ability to prevent neuronal loss during insults. Many neurotrophins have been used in studies to demonstrate neuroprotection against an array of acute insults. Neurotrophins of interest include, but are not limited to, nerve growth factor (NGF), glial-derived neurotrophic factor (GDNF), hepatocyte growth factor (HGF), brain-derived neurotrophic factor (BDNF), ciliary neurotrophic factor (CNTF), fibroblast growth factor 2 (FGF-2), neurotrophin 3 (NT3), and transforming growth factor β (TGF-β).

Reduction of hyperexcitation. In the case of seizures, hyperexcitation is the initiating event, whereas for hypoxia-ischaemia and hypoglycaemia, it is secondary to the decay of the resting potential that is caused by energy depletion. Outwardly rectifying potassium channels play a central role in terminating the action potential, returning the neuron to its negative resting potential, and maintenance after hyperpolarization. As such, THI delivery of these channels reduces hyperexcitation. Potassium channels that only open during hyperexcitation are of particular interest, including ATP-gated channels, which are opened by ATP depletion, channels that are activated by excessive cytosolic calcium, or voltage-dependent channels, which are opened by hyperexcitation.

Hyperexcitation can also be countered by the enhancement of hyperpolarizing chloride conductance. A related method enhances the effects of the inhibitory neurotransmitter GABA (-aminobutyric acid), which works by activating chloride conductance. Delivery of glutamic acid decarboxylase (GAD), the rate-limiting enzyme in the GABA synthetic pathway, increases GABA synthesis, thereby reducing insult-induced hyperexcitability and decreasing neurotoxicity. Alternatively, GABA receptor subunits can be overexpressed.

Targeting the energetic consequences of acute insults. Acute insults generate energy crises by disrupting energy production, as in hypoglycaemia or hypoxia-ischaemia, or by pathologically consuming energy, as in sustained seizures. This energy disruption is central to necrotic death, the consequences of which bias towards increased glutamate release, impaired glutamate uptake, increased calcium mobilization and impaired calcium extrusion. As such, delivery of a glucose transporter is of interest. During acute insults, sympathetic and adrenocortical activation increases circulating glucose and cerebral perfusion rates, and transiently opens the blood brain barrier. Furthermore, cerebral glucose transporter expression is upregulated as a defensive compensation post-insult. Overexpression of the Glut1 glucose transporter has been found to protect cultured hippocampal, septal and spinal neurons from excitotoxic or metabolic injuries. Moreover, expression of the transporter in vivo decreases hippocampal or striatal damage after seizure or ischaemia.

Targeting calcium excess. Extreme insults cause a disruption of ionic gradients that is sufficient to reverse the direction of calcium transporters, thereby increasing calcium influx. Delivery of a calcium binding protein protects against this type of insult. Binding proteins of interest include calbindin D28K. Calbindin overexpression protects PC12 cells from excitotoxins, pro-oxidants and serum withdrawal. Moreover, overexpression protects cultured hippocampal neurons from β-amyloid, excitotoxins, hypoglycaemia and cyanide, and reduces excitotoxin-induced declines in metabolism, ATP concentrations and mitochondrial potential. Overexpression of this protein also protects the hippocampus from excitotoxins, antimetabolites and ischaemia, and protects the striatum from infarct.

Targeting reactive oxygen species (ROS) accumulation. Superoxide, an ROS that is readily generated during insults, is converted to the less potent hydrogen peroxide by superoxide dismutases (SOD). As such, delivery of SOD and either catalase or glurathione peroxidase to the site of injury using THI is beneficial.

Augmenting heat shock response. The induction of heat-shock responses by insults, and the protective role of heat-shock proteins (HSPs) are well known. Examples of HSPs that protect neuronal cells include Hsp70, Hsp27 and Hsp54.

Blocking apoptosis. Numerous proteins that function to block apoptotic processes are known in the art. For example, overexpression of Bcl2 (B-cell leukaemia/lymphoma 2), a mammalian anti-apoptotic protein, decreases neuronal death that has been induced by excitotoxins, pro-oxidants and hypoglycaemia, and in cortical cultures it protects against excitotoxins. It also protects hypothalamic tumor cell lines against anoxia/aglycemia, and neuronal NT2N cell lines against hypoxia. In vivo, it decreases neurotoxicity that is caused by transient or permanent ischaemia in the cortex and striatum, seizure or global ischaemia in the hippocampus, nerve root crush, spinal cord contusion, or axotomy of photoreceptors.

As another example, Bclxl is functionally related to Bcl2, and its overexpression decreases the neuronal loss that is routinely seen in cortical cultures in the absence of insult. It also reduces axotomy-induced septal neurotoxicity, and apoptosis that has been induced by potassium withdrawal in cerebellar granule cultures.

Members of the inhibitor of apoptosis protein (IAP) family are also of interest. Overexpression of neuronal-apoptosis-inhibitory protein (NAIP), human IAP1, IAP2 or X chromosome IAP (XIAP) prevents neuronal apoptosis in primary cultures. Furthermore, XIAP protects against global ischaemic damage to the hippocampus.

The serine-threonine kinase Akt mediates many of the neuroprotective actions of growth factors, and blocks nitric oxide-induced apoptosis, caspase and Bax (Bcl2-associated X protein) activation, and Bcl2 depletion in hippocampal neurons. Also of interest are dominant negative mutants of Hif1 or Apaf1, which dominant-negative proteins counteract the pro-apoptotic effects of their native counterparts.

Viral anti-apoptotic genes also find use in the subject invention. As is known in the art, one host defense against viral infection is to trigger apoptosis in infected cells, thereby blocking viral replication, and numerous viruses have co-evolved anti-apoptotic mechanisms. Consonant with this, overexpression of the genes that encode the caspase inhibitors CrmA of the cowpox virus, or p35 of baculovirus, protects cultured hippocampal neurons from excitotoxic and metabolic insults, and also protects the hippocampus from seizure.

### Administration of Carrier Immunocytes

Carrier immunocytes are administered to a subject in any physiologically acceptable medium, normally intravascularly, although they may also be introduced into the targeted site, where the cells home to the site of inflammation. The number of cells administered may vary widely depending on the nature of the CNS disease being treated and/or the therapeutic gene of interest. As such, the number of carrier immunocytes administered can include at least about 1×10⁵ carrier immunocytes, at least about 1×10⁵ carrier immunocytes, at least about 1×10⁷ carrier immunocytes, at least about 1×10⁸ carrier immunocytes, at least about 1×10⁹ carrier immunocytes or more. In addition, the carrier immunocytes can be administered in a single dose or at timed intervals.

In the methods of the invention, cells are transferred to a recipient in any physiologically acceptable excipient comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996. Choice of the cellular excipient and any accompanying elements of the composition will be adapted in accordance with the route and device used for administration. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium.

### Disease Conditions

“Neurologic disorder” is defined here and in the claims as a disorder in which dysfunction of neurons occurs either in the peripheral nervous system or in the central nervous system. Examples of neurologic disorders include: chronic neurodegenerative diseases such as Alzheimer's disease, Parkinson's disease, Huntington's chorea, diabetic peripheral neuropathy, multiple sclerosis, amyotrophic lateral sclerosis; psychiatric diseases and acute neurodegenerative disorders including: stroke, traumatic brain injury, peripheral nerve damage, hypoglycemia, spinal cord injury, epilepsy, anoxia and hypoxia. Acute conditions are of particular interest, because of their prevalence and current lack of effective treatments.

Stroke is the third ranking cause of death in the United States, and accounts for half of neurology inpatients. Depending on the area of the brain that is damaged, a stroke can cause coma, paralysis, speech problems and dementia. The five major causes of cerebral infarction are vascular thrombosis, cerebral embolism, hypotension, hypertensive hemorrhage, and anoxia/hypoxia. In each of these cases, impaired energy and metabolite delivery to the affected brain region can result in significant neuronal cell loss.

The brain requires glucose and oxygen to maintain neuronal metabolism and function. Hypoxia refers to inadequate delivery of oxygen to the brain, and ischemia results from insufficient cerebral blood flow. The consequences of cerebral ischemia depend on the degree and duration of reduced cerebral blood flow. Neurons can tolerate ischemia for 30-60 minutes, but perfusion must be reestablished before 3-6 hours of ischemia have elapsed. Neuronal damage can be less severe and reversible if flow is restored within a few hours, providing a window of opportunity for intervention.

If flow is not reestablished to the ischemic area, a series of metabolic processes ensue. The neurons become depleted of ATP and switch over to anaerobic glycolysis (Yamane et al. (2000) J Neurosci Methods 103(2):163-71). Lactate accumulates and the intracellular pH decreases. Without an adequate supply of ATP, membrane ion pumps fail. There is an influx of sodium, water, and calcium into the cell. The excess calcium is detrimental to cell function and contributes to membrane lysis. Cessation of mitochondrial function signals neuronal death (Reichert et al. (2001) J Neurosci. 21(17):6608-16). The astrocytes and oligodendroglia are slightly more resistant to ischemia, but their demise follows shortly if blood flow is not restored (Sochocka et al. (1994) Brain Res 638(1-2):21-8)

Acute inflammatory reactions to brain ischemia are causally related to brain damage. The inflammatory condition consists of cells (neutrophils at the onset and later monocytes) and mediators (cytokines, chemokines, others). Upregulation of proinflammatory cytokines, chemokines and endothelial-leukocyte adhesion molecules in the brain follow soon after an ischemic insult and at a time when the cellular component is evolving.

The term “stroke” broadly refers to the development of neurological deficits associated with impaired blood flow regardless of cause. Potential causes include, but are not limited to, thrombosis, hemorrhage and embolism. Current methods for diagnosing stroke include symptom evaluation, medical history, chest X-ray, ECG (electrical heart activity), EEG (brain nerve cell activity), CAT scan to assess brain damage and MRI to obtain internal body visuals. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardiac arrest, cardiogenic shock, septic shock, head trauma, spinal cord trauma, seizure, bleeding from a tumor, or other blood loss.

By “ischemic episode” is meant any circumstance that results in a deficient supply of blood to a tissue. Where the ischemia is associated with a stroke, it can be either global or focal ischemia, as defined below. The term “ischemic stroke” refers more specifically to a type of stroke that is of limited extent and caused due to blockage of blood flow. Cerebral ischemic episodes result from a deficiency in the blood supply to the brain. The spinal cord, which is also a part of the central nervous system, is equally susceptible to ischemia resulting from diminished blood flow.

By “focal ischemia,” as used herein in reference to the central nervous system, is meant the condition that results from the blockage of a single artery that supplies blood to the brain or spinal cord, resulting in damage to the cells in the territory supplied by that artery.

By “global ischemia,” as used herein in reference to the central nervous system, is meant the condition that results from a general diminution of blood flow to the entire brain, forebrain, or spinal cord, which causes the death of neurons in selectively vulnerable regions throughout these tissues. The pathology in each of these cases is quite different, as are the clinical correlates. Models of focal ischemia apply to patients with focal cerebral infarction, while models of global ischemia are analogous to cardiac arrest, and other causes of systemic hypotension.

Stroke can be modeled in animals, such as the rat, by occluding certain cerebral arteries that prevent blood from flowing into particular regions of the brain, then releasing the occlusion and permitting blood to flow back into that region of the brain (reperfusion). Certain regions of the brain are particularly sensitive to this type of ischemic insult. The hippocampus, and more specifically the CA1 region of the hippocampus, is mostly affected in global models. However, the precise region that is directly affected is dictated by the location of the blockage and duration of ischemia prior to reperfusion. With increasing periods of global occlusion, cortical regions and the striatum are damaged besides the hippocampus (Lipton (1999) Physiol. Rev. 79: 1431-1568). Use of the methods of the invention is contemplated with animal models for neurodegeneration.

Traumatic brain injury (TBI) is often associated with cerebral concussion, which is a traumatically induced derangement of the nervous system, characterized clinically by immediate and transient impairment of consciousness and is generally not associated with remarkable gross anatomical changes. TBI is known to be a biphasic process. The first phase, the excitatory phase, occurs immediately upon injury. During this phase there is great neuronal excitation due to the trauma. Following the excitatory phase is the recovery phase, during which the neuronal excitation has abated and the job of repair has begun. Most often with traumatic brain injury, the excitatory phase is associated with increased intracranial pressure (ICP), with fluctuations of ICP over several days or more.

The methods of the invention are also useful for treatment of injuries to the central nervous system that are caused by mechanical forces, such as a blow to the head or spine. Trauma can involve a tissue insult such as an abrasion, incision, contusion, puncture, compression, etc., such as can arise from traumatic contact of a foreign object with any locus of or appurtenant to the head, neck, or vertebral column. Other forms of traumatic injury can arise from constriction or compression of CNS tissue by an inappropriate accumulation of fluid (for example, a blockade or dysfunction of normal cerebrospinal fluid or vitreous humor fluid production, turnover, or volume regulation, or a subdural or intracranial hematoma or edema). Similarly, traumatic constriction or compression can arise from the presence of a mass of abnormal tissue, such as a metastatic or primary tumor.

The compositions and methods of the invention find use in the treatment of mammals, such as human patients, suffering from neural injury or disease. The methods also find use in the treatment of non-human mammals, e.g. sport animals, e.g. horses; farm animals, e.g. cows, goats, pigs, etc.; pet animals, e.g. cats, dogs, etc., and laboratory animals, e.g. rats, mice, rabbits, etc. The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e.; causing regression of the disease. The therapeutic agent is usually administered after the onset of disease or injury. The treatment of ongoing disease, where the treatment stabilizes or reduces the undesirable clinical symptoms of the patient, is of particular interest.

An effective dose is the dose that, when administered for a suitable period of time, usually at least about one week, and may be about two weeks, or more, up to a period of about 4 weeks, 8 weeks, or longer will evidence an increase in the neurologic function, or maintenance of function that would otherwise be lost.

THI has immediate application for treatment against recurrent seizures, transient ischemia attacks, and stroke associated with heart bypass surgeries, as well as traumatic brain injury. It is important to note that nearly 1 in 5 patients undergoing heart bypass surgery with valve repair suffer a stroke during the surgery or in recovery. THI is also useful for the treatment of any acute or long-term condition that involves localized inflammation in the CNS. THI is also useful in the elucidation of disease and/or physiological processes that exists within localized regions of inflammation, for example by allowing the directed introduction of a gene or protein of interest to a targeted site of inflammation.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All publications and patent applications cited in this specification are herein incorporated by reference as if each individual publication or patent application were specifically and individually indicated to be incorporated by reference.

### Experimental

### EXAMPLE 1

### DCs Generated In Vitro Migrate to Site of CNS Lesion

DCs for this experiment were derived from bone marrow precursor cells, as described previously (Grauer et al. (2002) Histochem. Cell Biol. 117(4): 351-62). Briefly, cell isolates from the femur of inbred, male Lewis rats were cultured in nutrient-rich media containing interleukin-4 (IL-4), granulocyte/macrophage-colony stimulating factor (GM-CSF), and Flt-3 ligand. Under these conditions, DCs form semi-adherent cell aggregates, which are further enriched by removal of non-adherent lymphocytes.

After seven days, resultant DC content was measured by flow cytometry. Cultured DCs were scored for expression of MHC class II (OX6), integrin-α-X (CD11c), and α-E2 integrin (OX62) using monoclonal antibodies, the expression of maturation-indicative co-stimulatory surface molecules CD80 and CD86. For each antibody, matched isotype controls were run in parallel to account for non-specific binding. In addition to analysis of cell surface marker expression, DC cell cultures were assayed for viability by propidium iodide (P.I.) staining, where a high level of P.I. incorporation is indicative of DNA fragmentation and cell death. Aside from B lymphocytes, DCs are among the only immune cells that constitutively express OX6 (Banchereau et al. (2000) Annu. Rev. Immunol. 18: 767-811), and are the only cells currently known to express OX62 (Muthana et al. (2004) J. Immunol. Methods 294(1-2): 165-79).

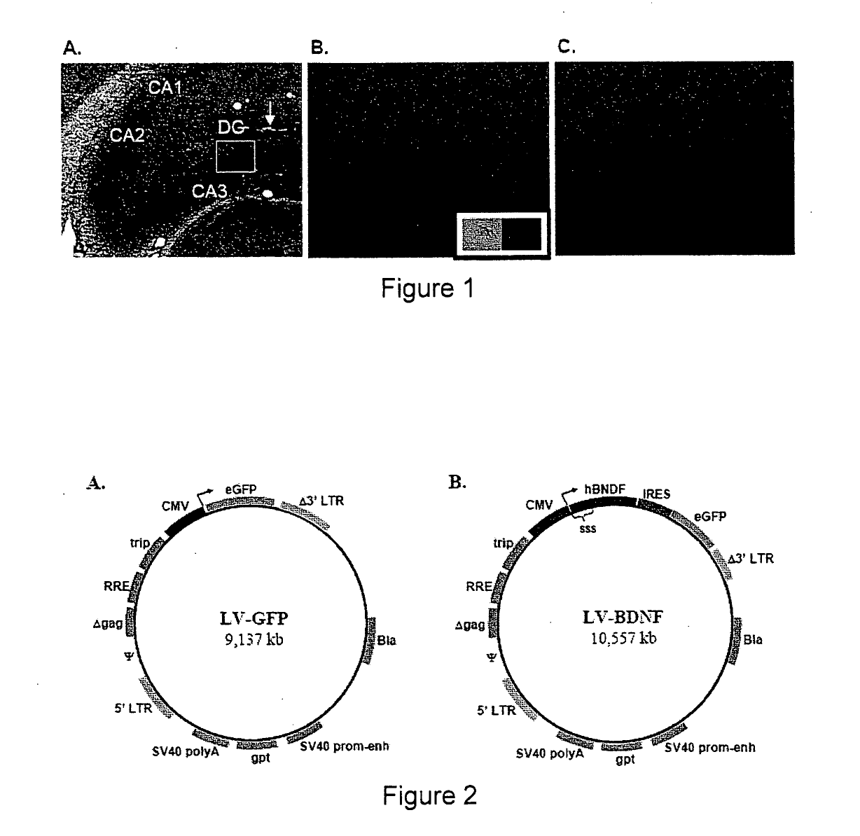

Endogenous DCs expressing OX6, OX62 and CD11c localize to the site of infarct as soon as one hour after permanent middle cerebral artery occlusion (pMCAO) in rats, indicating that these cells respond to CNS-specific inflammation (Kostulas et al. (2002) Stroke 33(4): 1129-34). As shown in FIG. 1, fluorescently labeled DCs generated by the above conditions migrate to the site of a KA-induced hippocampal lesion within six hours of administeration via the tail vein in a rat model (shown in FIG. 1B). Therefore, DCs generated and manipulated in vitro are capable of accessing an inflammatory site in the CNS similar to endogenous DCs.

### EXAMPLE 2

### Lentiviral Vectors and DC Infection/Transduction

Lentiviral vectors (LV) that express the reporter gene, eGFP (LV-GFP) are employed to determine optimal transduction conditions of DC cell cultures (FIG. 2A). LV-GFP vector is prepared as previously described (Kafri et al, (1999) J. Virol. 73(1): 576-84). Production of LV particles occurs in 293T kidney cells, allowing in trans expression of LV packaging proteins, the VSV-G envelope protein, and the actual transfer vector. By this approach, resultant LV particles only carry the transfer vector, which contains the chosen transgene and a minimal set of LV genes required for reverse transcription and nuclear integration (FIG. 2A). Omission of LV packaging proteins and the VSV-G envelope protein limits the activity of LV particles to one round of DC infection and transgene integration. In addition, the transfer vector is made ‘self-inactivating’ by a deletion in the U3 region of the 3′ long terminal repeat (LTR) to minimize the risk of interference with DC gene expression.

DC cultures are infected with LV-GFP particles on culture day 1, 4, or 6 with a multiplicity of infection (MOI) ranging from 10-15, since similar conditions yield a transduction efficiency of 95% for murine DCs (Breckpot et al. (2003) J. Gene. Med. 5(8): 654-67). The percentage of GFP-positive cells in infected and non-infected DC cultures is measured by flow cytometry on culture day 7, representing the intended day of use of the infected DCs. Co-stain of infected cell cultures with the DC-specific markers, CD11c, OX6, and OX62, as well as the monocyte/macrophage marker, CD11b (OX42), are used to characterize the GFP-positive cells. Infected and non-infected DC cultures are assayed for overall viability by P.I. staining. By varying the onset and duration of LV exposure, as well as MOI, it will be possible to identify the conditions that produce the highest yield of stably transduced DCs.

Starting with the optimal conditions for LV-GFP, a second LV vector containing BDNF (LV-BDNF) is assayed for DC transduction efficiency. Construct design includes an internal ribosomal entry site (IRES) in between BDNF and eGFP, allowing expression of both genes by the early immediate cytomegalovirus (CMV) promoter (FIG. 2B). For subsequent detection and quantification, BDNF is ligated to a myc tag and assayed for unperturbed protein structure (e.g. by two-dimensional sodium dodecyl sulfate polyacrylamide gel electrophoresis). Since eGFP is retained in LV-BDNF, the percentage of GFP-positive cells is a common measure of transduction efficiency. As before, relative CD11c, OX6, and OX62 provide a measure of DC-specific transduction, and P.I. incorporation shows resultant cell viability.

The highest transduction efficiency may occur through LV infection at the bone-marrow precursor cell stage (culture day 1), since the integrated transgene is carried by successive generations of each infected cell. However, constitutive transgene expression by the CMV promoter draws significant energy from transduced cells, leading to impaired cell proliferation and viability.

### EXAMPLE 3

### Assay for In Vitro Secretion of BDNF by Transduced DCs

BDNF normally exists as an extracellular protein, and its cDNA contains an endogenous signal secretory sequence. Consequently, LV-mediated integration of unmodified BDNF allows proper secretion from transduced DCs. LV vectors are designed to contain myc-tagged human BDNF followed by an IRES and eGFP (LV-BDNF, FIG. 2B).

DC cultures are transduced with LV-BDNF or LV-GFP according to the methods described above. Flow cytometry is used to assay the percentage of GFP-positive DCs, thus providing a measure of transduction efficiency. At 0, 12 and 24, and 48 hours post-transduction, the supernatant from DC cultures is collected and used for sandwich ELISA. Briefly, each sample of DC supernatant is added to wells coated with a monoclonal antibody for BDNF. After 1 hour of incubation, a second, biotinylated antibody for BDNF is added to each well and assayed for relative absorbance at the appropriate wavelength. Purified BDNF protein and DCs transduced with LV-GFP serves as positive and negative controls, respectively.

Relative absorbance for DC supernatant is matched with known concentrations of purified BDNF protein to approximate the amount of BDNF secreted by transduced DCs. Results from at least three separate experiments are combined to obtain an average measurement of transgene protein secretion.

### EXAMPLE 4

### Assay for In Vivo Secretion of BDNF

BDNF secretion by carrier DCs at the site of hippocampal lesion is assessed in an animal model. Following infusion of KA into the left dentate gyrus, animals are injected with DCs containing LV-BDNF, DCs containing LV-GFP, or vehicle. Animals are sacrificed at the time point of maximal DC localization to the hippocampal lesion. Brains are fixed by transcardial perfusion with paraformaldehyde and sectioned for immunohistochemistry and cresyl violet staining. Every fifth section (10 μm increments) is used for immunohistochemistry for α-myc and GFP. Every sixth section is subject to cresyl violet stain and used for lesion quantification.

The presence of BDNF protein is compared for animals injected with DCs containing LV-BDNF, DCs containing LV-GFP, or vehicle. The presence of GFP-positive cells confirms successful localization of carrier DCs, and segregation of a-myc signal from GFP-positive cells supports successful secretion of BDNF.

Incorporation of the IL-2 secretory signal sequence has been shown to enhance secretion of FGF2 from transduced neuronal cells (Matsuoka et al., (2000) Neuroreport 11(9):2001-6). In addition, the IL-2 signal sequence should favor DC-specific secretion, since IL-2 is commonly produced by DCs. Fusion of the IL-2 signal sequence to BDNF will not affect protein function as the secretory signal sequence is cleaved upon translocation to the ER. Further, incorporation of the IL-2 signal sequence may enhance BDNF secretion in vivo, specifically upon arrival of transduced DCs to the site of hippocampal lesion. DCs normally upregulate production and secretion of IL-2 in response to inflammation, thus providing a potential mechanism for targeted transgene secretion.

### EXAMPLE 5

### Neuroprotection of Lesioned Hippocampus by THI-Mediated Delivery of BDNF

Quantification of the neuroprotection provided by BDNF-transduced DCs is assessed during status epilepticus. Cresyl violet-stained sections obtained in are used to quantify the CA3 lesion in animals injected with LV-BDNF DCs, LV-GFP DCs, or vehicle. For each animal, volume measurements of intact CA3 are obtained for both hemispheres (KA-infused and control) using bright field microscopy and the imaging program, MetaMorph (Universal Imaging Corporation). The total volume of lesioned CA3 in the left hippocampus of each animal is expressed as a percentage of the control, contralateral hemisphere. Percent CA3 lesion is averaged within each group to allow statistical comparison between experimental and control scenarios.

For comparison, a separate set of animals is given an iv injection of hBDNF protein fused to a monoclonal antibody for the transferrin receptor (bdnf-ffn) immediately after hippocampal infusion of KA. A 50 μg dose of this chimeric protein significantly reduces infarct size following pMCAO in rats, but has not been tested for KA-induced status epilepticus. Initially, KA-infused animals are given 25 μg, 50 μg, or 100 μg of bdnf-tfn (n=4/dose) and quantified for CA3 lesion 24 hours after KA delivery.

Comparison of animals injected with LV-BDNF DCs versus those that receive vehicle assesses the neuroprotection provided by THI. Next, this value is compared to the neuroprotection conferred by iv delivery of bdnf-tfn in order to evaluate the two forms of CNS therapy. Additionally, comparison of CA3 lesion for animals that receive LV-GFP DCs versus those that receive vehicle reveals any neuron death associated with the DC carrier system.

### EXAMPLE 6

### Reduction of Inflammation at Site of Hippocampal Lesion Using THI

Initially, inflammation is measured within the context of status epilepticus and THI-mediated delivery of BDNF. Experimental and control groups are set up the same as above, where animals receive an injection of LV-BDNF DCs, LV-GFP DCs, non-transduced DCs, red blood cells (RBC), or vehicle following hippocampal infusion of KA. For each animal, brain sections are collected at the time of earliest DC localization, the latest time of DC localization, and the midpoint between the two. Sections are stained for the macrophage/monocyte/microglial-specific marker, CD11b, where segregation from GFP signal is indicative of endogenous immune cells. Comparison of animals subjected to THI versus those that receive vehicle estimates the recruitment of immune cells directly associated with THI. Further comparison with animals that receive non-transduced DCs or RBCs dissociates neuroinflammation that results from LV or the cell transfer process, respectively.

The level of pro-inflammatory cytokines at the site of lesion is assessed in control and THI-treated animals. Whole cell extracts are collected from the lesioned hippocampus of animals injected with LV-BDNF DCs, LV-GFP DCs, non-transduced DCs, RBCs, or vehicle at the same time points. Reverse transcriptase polymerase chain reaction (RT-PCR) determines mRNA levels of the key pro-inflammatory cytokines, IL-1, IL-2, IL-6, and TNF (tumor necrosis factor) and the chemokines, IL-8, MCP-1, MIP-2 (macrophage inflammatory protein-2), and MIP-1α. This set of cytokines and chemokines is commonly attributed to inflammatory damage in neurological disorders (Watson et al. (2005) Hum. Gene Ther. 16(1): 49-56), and MCP-1 and MIP-2 are induced in the rat hippocampus following infusion of KA (Kalehua et al. (2004) Exp. Cell Res. 297(1): 197-211). Comparison of cytokine/chemokine levels for animals given transduced DCs versus those that receive vehicle provides a second, independent measurement of the reduction of neuroinflammation associated with THI.

What is claimed is:

1. A method of treating a subject having a central nervous system (CNS) lesion, said method comprising:
obtaining a population of subject-compatible immunocytes;
genetically engineering said immunocytes to express and secrete a therapeutic protein; and
administering said genetically engineered immunocytes (or carrier immunocytes) into said subject;
wherein said carrier immunocytes localize to said CNS lesion and secrete said therapeutic protein.

2. The method according to claim 1, wherein said immunocytes are antigen presenting cells.

3. The method according to claim 2, wherein said antigen-presenting cells are dendritic cells (DCs).

4. The method according to claim 1, wherein said immunocytes are autologous.

5. The method according to claim 1, wherein said immunocytes are allogeneic.

6. The method according to claim 1, wherein said immunocytes are generated in vitro from precursor cells.

7. The method according to claim 6, wherein said precursors are derived from lymphoid tissue selected from the group consisting of bone marrow, spleen, lymph nodes, peripheral blood, and cord blood.

8. The method according to claim 1, wherein said immunocytes are genetically engineered using a viral vector comprising an expression cassette encoding said therapeutic protein.

9. The method according to claim 8, wherein said viral vector is a lentiviral vector.

10. The method according to claim 1, wherein said therapeutic protein inhibits neuronal necrosis and/or apoptosis at said CNS lesion in said subject.

11. The method according to claim 10, wherein said therapeutic protein blocks neuronal apoptosis.

12. The method according to claim 10, wherein said therapeutic protein blocks inflammatory signals.

13. The method according to claim 10, wherein said therapeutic protein is a neurotrophin.

14. The method according to claim 13, wherein said neurotrophin is brain-derived neurotrophic factor (BDNF).

15. The method according to claim 1, wherein said therapeutic protein acts extracellularly at said CNS lesion.

16. The method according to claim 1, wherein said therapeutic protein acts intracellularly at said CNS lesion.

17. The method according to claim 16, wherein said therapeutic protein comprises a protein translocation domain (PTD).

18. The method according to claim 1, wherein said CNS lesion is chosen from stroke, concussive head trauma, transient ischemic attacks, hypoglycemia, seizure, Alzheimer's disease, multiple sclerosis, and Parkinson's disease.

19. The method according to claim 1, wherein said carrier immunocytes are administered to the bloodstream of said subject.

20. The method according to claim 1, further comprising freezing and storing said carrier immunocytes prior to therapeutic use.
